(19) United States
(12) Patent Application Publication
(10) Pub. No.: US 2017/0209311 A1
(43) Pub. Date: Jul. 27, 2017

(54) ABSORBENT ARTICLES WITH NON-ADHERENT MEMBER

(71) Applicant: Covidien LP, Mansfield, MA (US)

(72) Inventors: Anand Kanchagar, Beaverton, OR (US); Carlos Gutierrez, Rancho Santa Margarita, CA (US)

(21) Appl. No.: 15/411,477

(22) Filed: Jan. 20, 2017

Related U.S. Application Data
(60) Provisional application No. 62/281,783, filed on Jan. 22, 2016.

(51) Int. Cl. A61F 13/00; B32B 27/12; B32B 27/36; A61F 13/53; B32B 5/02

(52) U.S. Cl. CPC A61F 13/00029; A61F 13/53; A61F 13/00008; A61F 13/00046; A61F 13/00987; B32B 5/022; B32B 27/36; B32B 27/12; A61F 2013/00217; A61F 2013/530481; A61F 2013/00263; B32B 37/12

(57) ABSTRACT

Example absorbent articles including a non-adherent member comprising a polyester, a substrate adjacent the non-adherent member, and an absorbent member disposed between the non-adherent member and the substrate are disclosed. The non-adherent member defines a plurality of perforations extending through a thickness of the non-adherent member. Example techniques for forming example articles are disclosed.

This application claims the benefit of U.S. Provisional Application No. 62/281,783, filed Jan. 22, 2016, which is incorporated by reference herein in its entirety.

### TECHNICAL FIELD

The present disclosure generally relates to absorbent articles, for example, wound dressings.

### BACKGROUND

Some absorbent articles such as wound dressings include an absorbent material capable of absorbing fluids such as wound exudates. Retention of fluids in contact with skin, for example, near wounds, may reduce the effectiveness of wound dressings in promoting wound healing. Retained fluids may also reduce adhesion between absorbent articles and skin, leading to premature peeling off of the absorbent articles from a skin surface of a patient.

### SUMMARY

In some examples described herein, an absorbent article comprises a non-adherent member including a polyester. The non-adherent member may be configured to contact a skin surface of a patient during use of the absorbent article. The absorbent article further includes a substrate, and an absorbent member disposed between the non-adherent member and the substrate. A portion of the substrate may be adjacent the non-adherent member in some examples. The non-adherent member defines a plurality of perforations extending through a thickness of the non-adherent member. In some examples, the polyester may include polyethylene terephthalate.

One or more examples of the present disclosure pertain to an example technique of forming an absorbent article. The example technique includes securing an absorbent member to a substrate, and securing at least a portion of a non-adherent member including a polyester to at least a portion of the absorbent member. The non-adherent member defines a plurality of perforations extending through a thickness of the non-adherent member. In some examples, the polyester may include polyethylene terephthalate.

Clause 1: An absorbent article comprising: a non-adherent member comprising a polyester; a substrate adjacent the non-adherent member, wherein the non-adherent member defines a plurality of perforations extending through a thickness of the non-adherent member; and an absorbent member disposed between the non-adherent member and the substrate.

Clause 2: The absorbent article of clause 1, wherein the non-adherent member is configured to non-adheringly contact skin of a patient.

Clause 3: The absorbent article of clause 1 or 2, wherein the non-adherent member comprises a polymeric film.

Clause 4: The absorbent article of clause 3, wherein the polymeric film consists essentially of the polyester.

Clause 5: The absorbent article of any one of clauses 1 to 4, wherein the polyester comprises polyethylene terephthalate.

Clause 6: The absorbent article of any one of clauses 1 to 5, wherein the absorbent member comprises a superabsorbent material.

Clause 7: The absorbent article of any one of clauses 1 to 6, wherein the substrate is moisture vapor permeable.

Clause 8: The absorbent article of claim 7, wherein the substrate has a moisture vapor transmission rate of at least about 500 grams per meters squared per day.

Clause 9: The absorbent article of any one of clauses 1 to 8, wherein the substrate comprises a laminated member.

Clause 10: The absorbent article of clause 9, wherein the laminated member comprises a nonwoven layer and a supporting film.

Clause 11: A wound dressing comprising the absorbent article of any one of clauses 1 to 10.

Clause 12: A method of forming an absorbent article, the method comprising: securing an absorbent member to a substrate, wherein the non-adherent member defines a plurality of perforations extending through a thickness of the non-adherent member; and securing at least a portion of a non-adherent member comprising a polyester to at least a portion of the absorbent member.

Clause 13: The method of clause 12, further comprising securing at least another portion of the non-adherent member to at least a portion of the substrate.

Clause 14: The method of clause 12 or 13, wherein one or both of the securing the absorbent member or securing at least a portion of the non-adherent member comprises one or more of heat bonding, adhesive bonding, static bonding, or corona treatment.

Clause 15: The method of any one of clauses 12 to 13, wherein the non-adherent member comprises a polymeric film.

Clause 16: The method of clause 15, wherein the polymeric film consists essentially of the polyester.

Clause 17: The method of any one of clauses 12 to 16, wherein the polyester comprises polyethylene terephthalate.

Clause 18: The method of any one of clauses 12 to 17, wherein the substrate has a moisture vapor transmission rate of at least about 500 grams per meters squared day.

Clause 19: The method of any one of clauses 12 to 18, wherein the absorbent member comprises a superabsorbent material.

Clause 20: The method of any one of clauses 12 to 19, further comprising laminating a nonwoven layer to a supporting film to form the substrate.

### BRIEF DESCRIPTION OF THE DRAWINGS

FIG. 1 is a schematic and conceptual illustration showing a plan view of an example absorbent article according to the present disclosure.

FIG. 2 is a schematic and conceptual illustration showing a cross-sectional view of the absorbent article of FIG. 1.

FIG. 3 is a schematic and conceptual illustration showing a partially-exploded cross-sectional view of the absorbent article of FIG. 1.

FIG. 4 is a schematic and conceptual illustration showing a cross-sectional view of the absorbent article of FIG. 1, further including a backing layer.

The details of one or more aspects of the disclosure are set forth in the accompanying drawings and the description below. Other features, objects, and advantages of the techniques described in this disclosure will be apparent from the description and drawings, and from the claims.

### DETAILED DESCRIPTION

In the present disclosure, the terms “proximal” and “distal” indicate relative proximity to a reference surface. For example, “proximal” indicates a location relatively nearer the reference surface, while “distal” indicates a location relatively farther from the reference surface. In some examples, the reference surface may include skin to which example absorbent articles according to the disclosure may be configured to be applied. Thus, a proximal surface may be a surface configured to face toward skin, while a distal surface may be a surface configured to face away from skin when an absorbent article is placed on a skin surface of a patient such that an absorbent member of the absorbent article absorbs substances from the patient, such as wound exudate.

In some examples, an example absorbent article may include a non-adherent member comprising a polyester, a substrate adjacent the non-adherent member, and an absorbent member disposed between the non-adherent member and the substrate.

Absorbent articles may need to be sterilized before they can be applied to a skin surface of a patient, for example, using steam sterilization or ethylene oxide sterilization. Steam sterilization may be conducted at about 212° F., and ethylene oxide sterilization may be performed at about 180° F. However, not all materials can resist the elevated temperatures of the steam sterilization, or even the ethylene oxide sterilization. For example, some polymers may begin softening or melting at temperatures under 180° F. Polyesters have a relatively high softening and melting point compared to other polymers. For example, polyesters such as polyethylene terephthalates have a softening point of higher than about 400° F., for example, about 470° F. Using a polyester in the non-adherent member allows sterilization of the absorbent article at elevated temperatures, without degrading components of the absorbent article. This may allow users who do not have access to lower temperature sterilization techniques, or only have access to steam sterilization, to perform sterilization without damaging the absorbent article. Polyesters may also have additional advantages, for example, allowing the formation of relatively narrow perforations that permit passage of fluids and exudates, as described elsewhere in the disclosure.

In some examples, an example wound dressing including an example absorbent article according to the disclosure may be relatively thin and conformable, which may allow the wound dressing to be placed on regions of a person's body that are at risk of pressure ulcers.

In some examples, example absorbent articles according to the present disclosure may exhibit a high absorbent capacity a high moisture vapor permeability, or any combination thereof. The high moisture vapor permeability may allow the absorbent article to breathe, which may increase patient comfort and facilitate wound healing if the absorbent article is used as a wound dressing. Wound dressings including example absorbent articles described herein may be used for treating heavily exuding wounds as well as acute wounds.

An absorbent article described herein may include an absorbent member that absorbs relatively large amounts of wound fluids and holds the fluid under pressure, allowing the absorbent article to provide a moist wound healing environment. In some such examples, because the wound fluid is locked in the absorbent member, the wound edges and surrounding intact skin may have a reduced likelihood of becoming macerated. In some examples, wound dressings including example absorbent articles according to the disclosure may facilitate locking wound fluids in an absorbent member to control, e.g., reduce wound odor. Maintaining the moist environment may also prevent drying of fluids which may cause the absorbent article to dry and attach to a skin surface, for example, a wound surface, which may cause the absorbent article to be more difficult to remove from the attachment site, e.g., on a patient's skin surface.

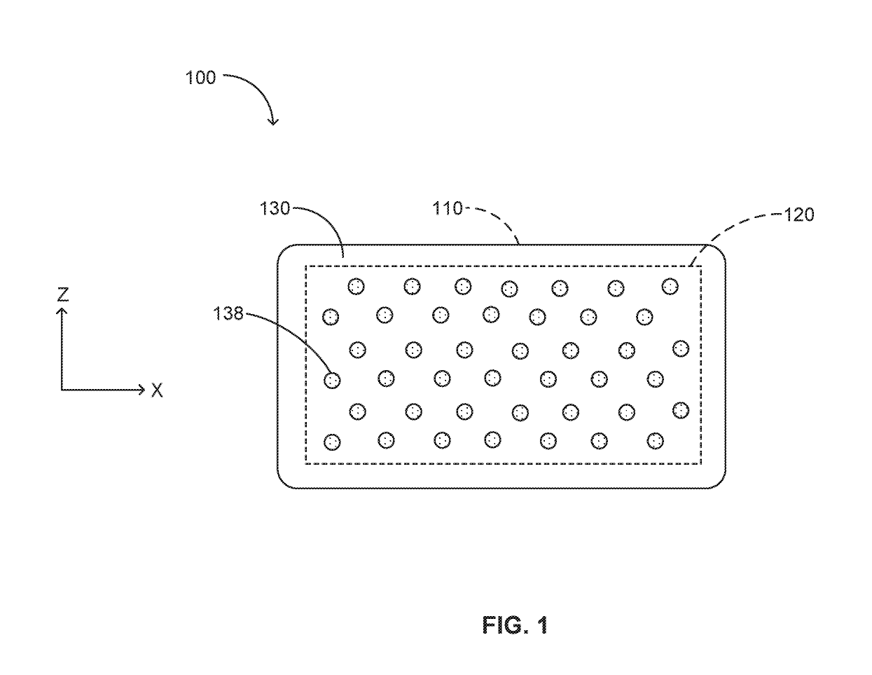

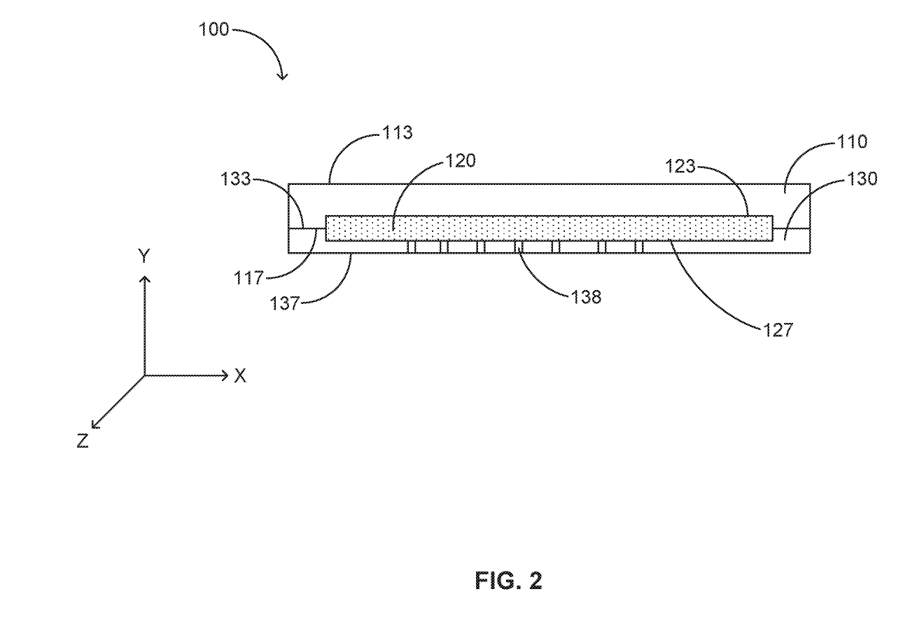

FIG. 1 is a schematic and conceptual illustration showing a plan view of an example absorbent article 100 according to the present disclosure. FIG. 2 is a schematic and conceptual illustration showing a cross-sectional view of absorbent article 100 of FIG. 1. Absorbent article 100 may be an article configured to be placed against a skin surface of a patient, for example, a wound dressing, a bed pad, a diaper, incontinence wear, or sanitary napkins. In some examples, example absorbent article 100 includes a substrate 110 defining a distal surface 113 and a proximal surface 117. Absorbent article 100 also includes an absorbent member 120 adjacent substrate 110. Absorbent member 120 defines a distal surface 123 and a proximal surface 127. In some examples, distal surface 123 of absorbent member 120 may be in contact with at least a portion of proximal surface 117 of substrate 110.

In the example shown in FIGS. 1 and 2, example absorbent article 100 further includes a non-adherent member 130. In some examples, non-adherent member 130 may define a plurality of perforations 138 through a thickness thereof, as described elsewhere in the disclosure. Non-adherent member 130 defines a distal surface 133 and a proximal surface 137. In some examples, non-adherent member 130 is disposed adjacent proximal surface 117 of substrate 110. For example, distal surface 133 of non-adherent member 130 may contact with at least a portion of proximal surface 117 of substrate 110. In some examples, distal surface 133 of non-adherent member 130 may optionally additionally contact proximal surface 127 of absorbent member 120. Thus, in some examples, absorbent member 120 may be disposed between proximal non-adherent member 130 and substrate 110, and enclosed (e.g., on all sides) by non-adherent member 130 and substrate 110, as shown in FIG. 2.

In some examples, a center of absorbent member 120 may be aligned with a center of substrate 110, as shown in FIGS. 1 and 2. In other examples, the center of absorbent member 120 may be offset from the center of substrate 110. In some examples, absorbent member 120 is sized to have a surface area that is less than the area of the proximal surface of substrate 110. Thus, in some examples, non-adherent member 130 can be optionally sized such that a portion of distal surface 133 of non-adherent member 130 contacts at least a portion of proximal surface 117 of the substrate 110. In some such examples, another portion of distal surface 133 of non-adherent member 130 may contact at least a portion of proximal surface 127 of absorbent member 120.

In some examples, substrate 110 may facilitate vapor permeability to accommodate fluids or vapor, for example, perspiration, wound exudates, or other bodily fluids or vapors, from a skin surface adjacent a wound to which absorbent article 100 may be applied. The vapor permeability of substrate 110 may help reduce heat and moisture vapor build up within absorbent article 100 by allowing the moisture vapor to exit article 100 through substrate 110. For example, absorbent article 100 may be applied to the skin surface such that non-adherent member 130 is proximal to and contacts the skin surface, while substrate 110 is distal from the skin surface. Thus, fluids from the skin surface may be absorbed across or through non-adherent member 130 into absorbent member 120. Vapor generated from the skin, for example, by body heat, may permeate away from the skin surface through substrate 110, ultimately reducing accumulation of respiration in absorbent article 100. Such permeation may also provide cooling to skin surface in the vicinity of absorbent article 100, for example, by wicking away relatively warm perspiration from the skin surface. Thus, substrate 110 may include a moisture vapor permeable member or otherwise be moisture vapor permeable to facilitate vapor permeability through absorbent article 100. In some examples, substrate member 110 has a moisture vapor transmission rate (MVTR) of at least about 500 grams per meters squared per 24 hours (g/m²/day, under ambient conditions or at room temperature. A moisture vapor transmission rate of at least about 500 g/m²/day may help prevent accumulation or pooling of body fluids or exudates adjacent or under absorbent article 100. In some examples, substrate member 110 may have a moisture vapor transmission rate greater than or equal to about 500 g/m²/day and less than or equal to about 5000 g/m²/day, for example, at least about 1000 g/m²/day.

In addition, in some examples, substrate 110 has a flexible configuration, and may facilitate conformability of absorbent article 100 when applied or adhered to curved or non-flat skin surfaces. In some examples, substrate 110 may serve as a supporting member having sufficient tensile strength to resist break or tearing in response to absorbent article 100 being removed from or applied on a skin surface.

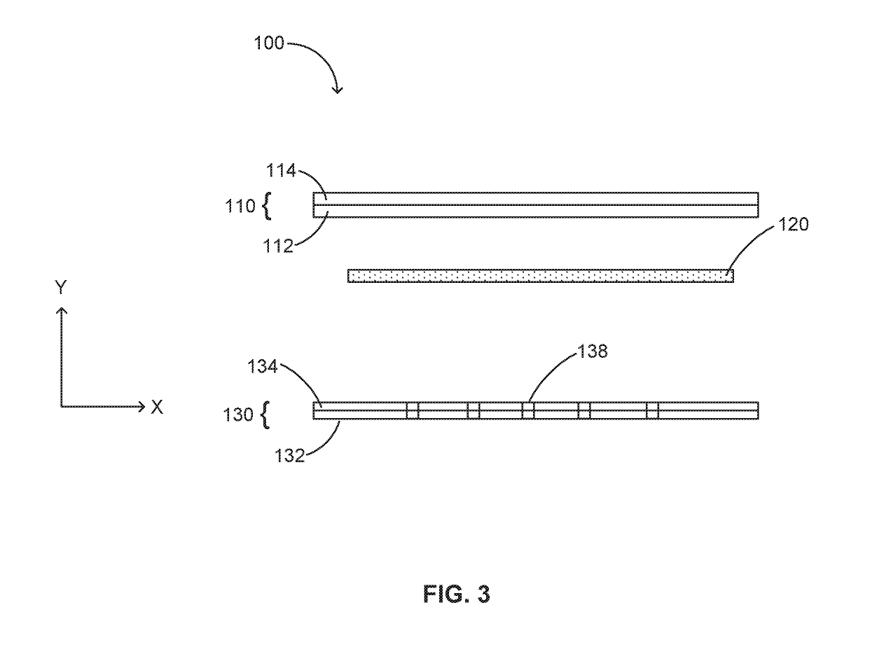

Substrate 110 may include a liquid impermeable member, for example, to prevent or reduce migration of fluids into absorbent article 100, to prevent or reduce migration of fluids contained within absorbent member 120 outside of article 10, or both. Such migration of fluids into absorbent article 100 may result from inadvertent external contact with liquids, for example, during washing, cleaning, or bathing of the patient to which the article is applied. The liquid impermeable member may also be moisture vapor permeable, for example, allowing release of vapor away from a skin surface through absorbent article 100 while reducing flow of external fluid through absorbent article 100 toward the skin surface. To provide both liquid impermeability and vapor permeability, in some examples, substrate 110 may include a laminated member, as shown in FIG. 3.

FIG. 3 is a schematic and conceptual illustration showing a partially-exploded cross-sectional view of absorbent article 100 of FIG. 1. The cross-section extends through some perforations of plurality of perforations 138. In the example shown in FIG. 3, substrate 110 includes a moisture vapor permeable laminate including a first layer 112 and a second layer 114 that is contiguous with and in contact with a distal surface of first layer 112. In some examples, substrate 110 can consist essentially of first layer 112 and second layer 114 that is contiguous with and in contact with first layer 112.

First layer 112 may be formed from any suitable material. For example, first layer 112 may include a breathable film, for example, as a vapor permeable polyethylene film. Second layer 114 may also be formed of any suitable material, and can be the same as or different than first layer 112. For example, second layer 114 may include a non-woven fabric, for example, a spun bond polypropylene material. The non-woven fabric of second layer 114 may be secured to first layer 112 by, for example, heat bonding, adhesive bonding, or static bonding. In some examples, securing first and second layers 112 and 114 together may be facilitated by corona treatment of the facing surface of one or both of first and second layers 112 and 114.

Absorbent member 120 may be formed from any suitable material that is configured to absorb and substantially retain fluids that contact a surface of absorbent article 100, for example, fluid exudates or respiration. For example, absorbent member 120 may include an absorbent material. The absorbent material may include foam, cellulosic material, non-woven materials, or any other suitable absorbent. In some examples, absorbent member 120 may include superabsorbent material, for example, in addition to, or instead of, a non-superabsorbent material. For example, the superabsorbent material may include superabsorbent particles (SAP), disposed, e.g., embedded, in a cellulosic matrix, such as tissue layers. Superabsorbent material is material that is capable of absorbing fluid several times its own volume. The use of superabsorbent material may allow absorbent member 120 to be relatively compact, for example, relatively thin in a direction across a thickness of absorbent article 100, for example, in a direction along the y-axis shown in FIGS. 2-4 (orthogonal x-y-z axes are shown in the figures for ease of description only), or relatively occupying a lower area relative to an area of absorbent article 100. For example, absorbent article 100 including superabsorbent material in absorbent member 120 may exhibit a low profile, avoiding problems associated with high profile wound dressings, for example, pressure ulcers. Thus, absorbent member 120 may include superabsorbent material such as SAP disposed within tissue layers that may be bonded together by an adhesive, by use of a latex sprayed thereon, or by effecting hydrogen bonding under application of high temperature and pressure onto the tissue layers. In some examples, absorbent member 120 may further include fluff pulp material in addition to or instead of the SAP. For example, absorbent member 120 may include SAP and fluff pulp disposed within tissue layers. In some examples, absorbent member 120 may consist essentially of SAP, fluff pulp, and a cellulosic matrix. In other specific configurations, absorbent member 120 can consist essentially of SAP and a cellulosic matrix.

In some examples, absorbent member 120 may include superabsorbent material, such as superabsorbent fibers, in a matrix of synthetic fibers. For example, absorbent member 120 may include superabsorbent fibers disposed within a matrix of polyester fibers that are needle-punched to form a nonwoven mat. In some examples, absorbent member 120 may further include fluff pulp material. For example, absorbent member 120 may include superabsorbent fibers, fluff pulp and polyester fibers needle-punched into a nonwoven layer. In some examples, absorbent member 120 may consist essentially of a needle-punched layer of superabsorbent fibers, fluff pulp, and polyester fibers. In other examples, absorbent member 120 may consist essentially of a needle-punched layer of superabsorbent fibers and polyester fibers.

While absorbent article 100 may include a unitary or single absorbent member 120, absorbent article 100 may include more than one absorbent member 120, for example, a plurality of absorbent members that may be spaced apart to allow for uniform transport and retention of fluid within absorbent article 100.

Non-adherent member 130 prevents a skin-facing or skin-contacting surface of absorbent article 100 from attaching or adhering to skin, and can be formed from any suitable material that exhibits low or no adherence to skin. In some examples, non-adherent member 130 includes at least one biologically inert polymer defining a proximal surface configured to contact skin. For example, non-adherent member 130 may include an inert polymeric film 132, as shown in FIG. 3. In some such examples, non-adherent member 130 may further include an adhesive layer 134 on a proximal surface or a distal surface of the film 132. In some examples, non-adherent member 130 may include or consist essentially of a structure, e.g., a single layer, adhesively secured to absorbent member 120 and, optionally, to the substrate 110. Thus, non-adherent member 130 may be applied on or secured to a surface of absorbent member 120 and, optionally on or to at least a portion of a surface of substrate 110 for example, on a portion of a surface of first layer 112 of substrate 110. In some examples, absorbent article 100 may be assembled by spraying or brushing an adhesive composition onto one or more interfacing or contacting surfaces of any one or more of substrate 110, absorbent member 120, and non-adherent member 130.

In some examples, non-adherent member 130 may define plurality of perforations 138 through a thickness thereof, as shown in FIGS. 1-4. For example, each perforation of plurality of perforations 138 or substantially all perforations of plurality of perforations 138 may extend through a thickness of a polyester layer of non-adherent member 130. Plurality of perforations 138 may one or more of facilitate skin respiration, wound exudate transport to absorbent member 120, or vapor permeability through absorbent article 100. Without perforations, polyester may not provide a desired moisture vapor permeability. For example, moisture vapor permeability of non-adherent member 130 including polyester without perforations may have a moisture vapor transmission rate of less than 500 grams per meters squared per day, for example, a moisture vapor transmission rate of zero. Without wishing to be bound by theory, the size, shape, and distribution of the plurality of perforations may control the rate of wound exudate absorption and the vapor permeability of the dressing.

Perforations 138 may have any suitable size and shape for imparting the desired moisture vapor permeability characteristics to non-adherent member 130. In some examples, plurality of perforations 138 may include perforations defining one or more of circular, square, triangular, elliptical, rectangular, polygonal, or any other closed boundaries. In some examples, plurality of perforations 138 may include perforations having an average diameter between about 0.1 millimeters (mm) and about 1 mm, such as about 0.635 mm (25 thousandths of an inch), measured in a direction perpendicular to the thickness of non-adherent member 130 (in an x-z plane, shown in FIGS. 1 and 2). Each perforation of the plurality of perforations 138 may have substantially the same diameter, accounting for manufacturing tolerances. In other examples, the plurality of perforations 138 may include perforations having a non-uniform range of diameters with an average diameter between about 0.1 mm and about 1 mm. If the average diameter of plurality of perforations 138 is too small, then the flow of fluids or exudates across non-adherent member 130 into absorbent member 120 may be impeded. If the average diameter of plurality of perforations 138 is too large, then drying fluids or exudates may induce adherence and cause non-adherent member 130 to become adherent. In some examples, non-adherent member may define at least about 300 holes per square inch (at least about 45 holes per square cm). While plurality of perforations 138 are depicted as being visible in FIGS. 1-4, in some examples, the size of plurality of perforations 138 may be smaller than a visible perception, and may not be perceptible to a visual inspection by a human eye.

In some examples, plurality of perforations 138 may include perforations substantially uniformly distributed throughout non-adherent member 130, for example, as shown in FIG. 1. In other examples, a higher density of uniformly distributed perforations of larger size, e.g., larger diameter perforations, may be disposed at regions of non-adherent member 130 overlaying absorbent member 120, compared to perforations at regions more directly overlaying substrate 110. Conversely, in some examples, certain areas of the non-adherent member 130, e.g., at areas overlaying the substrate 110, may define a lower density of perforations or even no perforations. In some examples, plurality of perforations may include perforations in a range of from about 55 perforations per square centimeter to about 62 perforations per square centimeter (e.g., about 371 holes per square inch). In some examples, the size and density of plurality of perforations 138 may provide an aggregate exposure area at least in a range of from about 10% to about 20%, e.g., at least about 15%, of a surface area of non-adherent member 130.

Adhesive layer 134 may include any suitable adhesive. For example, adhesive layer 134 may include a hypoallergenic adhesive, such as an acrylic pressure sensitive adhesive, and may be coated or applied onto an intermediate surface within absorbent article 100, for example, at a surface of polymeric film 132. In some examples, adhesive layer 134 may define an average thickness in a range of from about 0.025 mm (about 1 mil) thick to about 0.127 mm (about 5 mil), measured in a direction along the y-axis shown in FIGS. 2-4.

In some examples, polymeric film 132 may be sufficiently thin to provide conformability about a skin-contacting surface of absorbent article 100, while being sufficiently thick to resist tearing during use, for example, in contact with skin or wounds, or during removal therefrom. In some examples, film 132 may define an average thickness in a range of from about 0.025 mm (about 1 mil) to about 0.25 mm (10 mil). The average thickness may be determined in a direction normal to a major surface of absorbent article 100, for example, between a most proximal surface and a most distal surface of absorbent article 100, in a direction along the y-axis shown in FIGS. 2-4. In some examples, polymeric film 132 includes a polyester, for example, polyethylene terephthalate. In some examples, polymeric film 132 consists essentially of the polyester. Using a polyester may allow polymeric film 132 to be non-adherent and conformable to a skin surface to which absorbent article 100 may be applied.

Example absorbent articles according to the disclosure may be applied or secured to a skin surface using any suitable technique. In some examples, absorbent article 100 may be secured by wrapping an adhesive tape or the like over absorbent article 100 (e.g., an outer periphery of absorbent article 100) and about a region of a skin surface. In addition, or instead, in some examples, a portion of substrate 110, for example, at least one peripheral region of substrate 110, may extend beyond the respective peripheries of one or both of absorbent member 120 and non-adherent member 130, such that the peripheral region of substrate 110 may be adhered to a skin surface. For example, any suitable adhesive capable of adhering absorbent article 100 to a skin surface may be applied to at least a portion of proximal surface 117 of substrate 110, for example, a peripheral region of proximal surface 117 of substrate 110. In some examples, the adhesive may be perforated to maintain the vapor permeability of substrate 110.

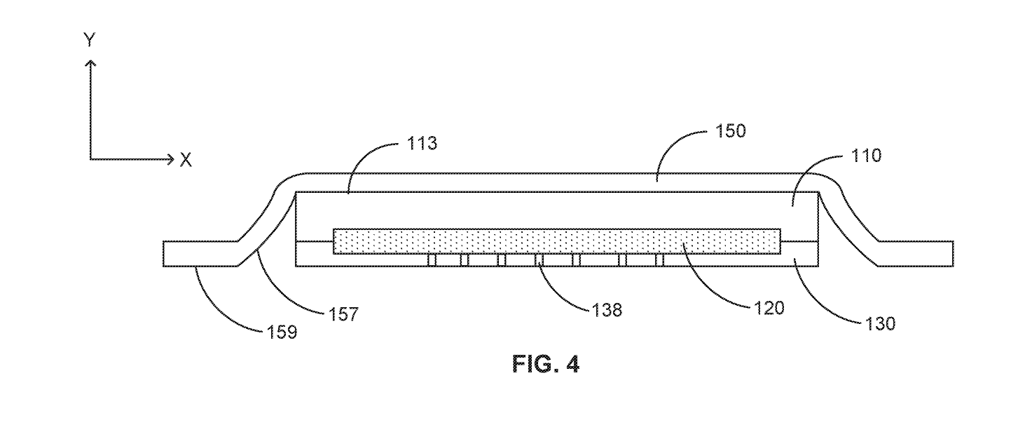

FIG. 4 is a schematic and conceptual illustration showing a cross-sectional view of the absorbent article of FIG. 1, further including a backing layer 150. The cross-section extends through some perforations of plurality of perforations 138. In the example shown in FIG. 4, backing layer 150 is distal to substrate 110. At least one peripheral region of backing layer 150, for example, peripheral region 159, extends beyond a peripheral region of one or both of absorbent member 120 and non-adherent member 130. Any suitable adhesive capable of adhering absorbent article 100 to a skin surface may be applied to at least a portion of a proximal surface 157 of backing layer 150, for example, peripheral region 159 of proximal surface 117 of backing layer 150. In some examples, absorbent article 100 including backing layer 150 or an extended substrate 110 may form an island dressing. Backing layer 150 may be perforated, or otherwise be moisture vapor permeable. In some examples, backing layer 150 has comparable or similar moisture vapor transmission rates as described with reference to substrate 110.

Example techniques according to the disclosure may be used to prepare (e.g., form or manufacture) example absorbent articles, for example, those described with reference to FIGS. 1 to 4. An example technique for forming absorbent article 100 may include securing absorbent member 120 to substrate 110, and securing at least a portion of non-adherent member 130 comprising a polyester to at least a portion of absorbent member 120. In some examples, the example technique may further include securing at least another portion of non-adherent member 130 to at least a portion of the substrate. In some examples, one or both of the securing absorbent member 120 or securing at least a portion of non-adherent member 130 includes one or more of heat bonding, adhesive bonding, static bonding, or corona treatment. In some examples, the example technique further includes laminating second layer 114, for example, including a nonwoven layer, first layer 112, for example, a supporting film, to form substrate 110. In some examples, backing layer 150 may be secured to substrate 110. While these example techniques may be used to prepare the example absorbent articles discussed with reference to FIGS. 1 to 4, example techniques according to the disclosure may also be used to prepare other example absorbent articles.

Thus, in some examples, example absorbent articles may include a non-adherent member including polyester configured to contact a skin surface having a wound. The polyester may be of medical grade. The non-adherent member including polyester may allow wound fluids or other secretions to pass through perforations and or otherwise get absorbed by the absorbent core. As the wound fluid dries, the non-adherent member may prevent the dressing from adhering to the wound bed.

Various examples have been described. These and other examples are within the scope of the following claims.

What is claimed is:

1. An absorbent article comprising:
a non-adherent member comprising a polyester, wherein the non-adherent member defines a plurality of perforations extending through a thickness of the non-adherent member;
a substrate adjacent the non-adherent member; and
an absorbent member disposed between the non-adherent member and the substrate.

2. The absorbent article of claim 1, wherein the non-adherent member is configured to non-adheringly contact skin of a patient.

3. The absorbent article of claim 1, wherein the non-adherent member comprises a polymeric film.

4. The absorbent article of claim 3, wherein the polymeric film consists essentially of the polyester.

5. The absorbent article of claim 1, wherein the polyester comprises polyethylene terephthalate.

6. The absorbent article of claim 1, wherein the absorbent member comprises a superabsorbent material.

7. The absorbent article of claim 6, wherein the substrate is moisture vapor permeable.

8. The absorbent article of claim 7, wherein the substrate has a moisture vapor transmission rate of at least about 500 grams per meters squared per day.

9. The absorbent article of claim 8, wherein the substrate comprises a laminated member.

10. The absorbent article of claim 9, wherein the laminated member comprises a nonwoven layer and a supporting film.

11. A wound dressing comprising the absorbent article of claim 1.

12. A method of forming an absorbent article, the method comprising:
securing an absorbent member to a substrate; and
securing at least a portion of a non-adherent member comprising a polyester to at least a portion of the absorbent member, wherein the non-adherent member defines a plurality of perforations extending through a thickness of the non-adherent member.

13. The method of claim 12, further comprising securing at least another portion of the non-adherent member to at least a portion of the substrate.

14. The method of claim 12, wherein one or both of the securing the absorbent member or securing at least a portion of the non-adherent member comprises one or more of heat bonding, adhesive bonding, static bonding, or corona treatment.

15. The method of claim 12, wherein the non-adherent member comprises a polymeric film.

16. The method of claim 15, wherein the polymeric film consists essentially of the polyester.

17. The method of claim 12, wherein the polyester comprises polyethylene terephthalate.

18. The method of claim 12, wherein the substrate has a moisture vapor transmission rate of at least about 500 grams per meters squared per day.

19. The method of claim 12, wherein the absorbent member comprises a superabsorbent material.

20. The method of claim 12, further comprising laminating a nonwoven layer to a supporting film to form the substrate.
